(19) United States
(12) Patent Application Publication
(10) Pub. No.: US 2024/0307666 A1
(43) Pub. Date: Sep. 19, 2024

(54) BALLOON FOR CATHETER AND METHODS RELATING TO THE SAME

(71) Applicant: TERUMO KABUSHIKI KAISHA, Tokyo (JP)

(72) Inventors: Daisuke SHIMADA, Fujinomiya Shizuoka (JP); Taku OKIYOSHI, Fujinomiya Shizuoka (JP); Takahiro NAKAHARA, Fujinomiya Shizuoka (JP)

(21) Appl. No.: 18/595,255

(22) Filed: Mar. 4, 2024

(30) Foreign Application Priority Data
Mar. 13, 2023 (JP) 2023-038919

(51) Int. Cl. A61M 25/10

(52) U.S. Cl. CPC A61M 25/1018; A61M 25/1029

(57) ABSTRACT

A method for determining that a balloon for a medical catheter is defective, includes, in a machining process of elongating a balloon base material with an elongation mechanism while applying pressure therein and heat thereto, monitoring a pressure value of the pressure and monitoring a movement amount of the mechanism or a time elapsed after a start of the process, detecting a start of expansion of the material based on the value, calculating: an elongation amount of the material immediately before the start of expansion based on the movement amount of the mechanism, or an elongation time from the start of the process to the start of expansion of the material based on the elapsed time, determining a pressure resistance of a balloon formed from the material based on the elongation amount or elongation time, and determining whether the balloon is defective based on the pressure resistance thereof.

### CROSS-REFERENCE TO RELATED APPLICATION(S)

This application is based on and claims the benefit of priority from Japanese patent application No. 2023-038919, filed Mar. 13, 2023, the entire contents of which are incorporated herein by reference.

### BACKGROUND

### Technical Field

Embodiments described herein relate generally to a method, a determination device, and a non-transitory computer readable medium that stores a program for determining that a defective balloon has been manufactured for a medical catheter, and a method for manufacturing a balloon for a medical catheter.

### Related Art

A medical catheter (hereinafter also referred to simply as a catheter) is used for diagnosis or treatment of a lesion present in a luminal organ such as a blood vessel or a vascular channel. As one type of medical catheter, a balloon catheter is used. A balloon catheter includes a flexible shaft to be inserted into the body of a patient, a rigid hub provided at a proximal end of the shaft, a distal end member disposed at the distal end side of the shaft, and a balloon for pushing and expanding a luminal organ.

The balloon of the balloon catheter is formed by setting a balloon base material formed with a material such as polyethylene, polypropylene, or silicone rubber in a mold, and pressurizing and inflating the balloon together with the mold at a predetermined pressure from the inside while heating the balloon base material.

In a medical procedure, such a balloon catheter is inserted into a luminal organ of a patient, and the balloon at the distal end is expanded by a fluid flowing through the shaft at the lesion. Therefore, balloons are required to be manufactured with a stable pressure resistance.

### SUMMARY

Embodiments of this disclosure provide a method, a determination device, and a non-transitory computer readable medium that stores a program for determining the pressure resistance of a balloon from balloon machining parameters and a method for manufacturing a balloon. Embodiments of this disclosure further provide a method for manufacturing a balloon for a medical catheter.

In one embodiment, a method for determining that a balloon for a medical catheter is defective, includes, in a machining process of elongating a balloon base material with an elongation mechanism while applying pressure therein and heat thereto, monitoring a pressure value of the pressure and monitoring a movement amount of the mechanism or a time elapsed after a start of the process, detecting a start of expansion of the material based on the value, calculating: an elongation amount of the material immediately before the start of expansion based on the movement amount of the mechanism, or an elongation time from the start of the process to the start of expansion of the material based on the elapsed time, determining a pressure resistance of a balloon formed from the material based on the elongation amount or elongation time, and determining whether the balloon is defective based on the pressure resistance thereof.

According to the present disclosure, it is possible to determine the pressure resistance of balloons in the manufacturing process to identify non-defective balloons having enough strengths within a predetermined range, and feedback a parameter such as the pressure for stabilizing the pressure resistance to the manufacturing process from the determination result, to enhance balloon catheter manufacturing accuracy.

### BRIEF DESCRIPTION OF THE DRAWINGS

FIG. 1 is a schematic view of a manufacturing system according to a first embodiment.

FIG. 2 is a hardware block diagram of a balloon molding machine of the manufacturing system.

FIG. 3 is a hardware block diagram of a determination device according to the first embodiment.

FIG. 4 is a flowchart illustrating a determination process to be performed by the determination device.

FIG. 5 is a flowchart illustrating the determination process to be performed by the determination device.

FIG. 6 illustrates an example of a graph displayed by the determination device.

FIG. 7 illustrates an example of a graph displayed by the determination device.

FIG. 8 is a graph plotting the relationship between the stretching distance before the start of expansion of a balloon and the actual pressure resistance thereof.

FIG. 9 is a graph illustrating a correlation between stretching distance before the start of expansion of a balloon and elongation of the balloon at its breakage.

FIG. 10 is a graph illustrating a correlation between stretching distance before the start of expansion of a balloon and Young's modulus.

FIGS. 11A and 11B are graphs each illustrating strength distribution depending on depth.

FIG. 12 is a flowchart illustrating a determination process to be performed by a determination device according to a second embodiment.

FIG. 13 is a flowchart illustrating the determination process to be performed by the determination device.

FIG. 14 is a flowchart illustrating the determination process to be performed by the determination device.

FIG. 15 is a diagram illustrating example contents of data stored in the determination device.

FIG. 16 is a schematic diagram of a trained model that outputs pressure resistance.

FIG. 17 is a flowchart illustrating a determination process using the trained model.

FIG. 18 is a flowchart illustrating the determination process using the trained model.

FIG. 19 is a flowchart illustrating a method for determining control parameters by the determination device.

FIG. 20 is a schematic diagram of a trained model that outputs control parameters.

FIG. 21 is a flowchart illustrating a method for determining the control parameters using the trained model.

FIG. 22 is a flowchart illustrating a method for manufacturing a balloon for a medical catheter.

FIG. 23 is a flowchart illustrating another method for manufacturing a balloon for a medical catheter.

### DETAILED DESCRIPTION

The present disclosure is specifically described below with reference to the drawings illustrating embodiments. In the following embodiments, systems for manufacturing a balloon of a medical catheter to which a determination method of the present disclosure is applied are described.

### First Embodiment

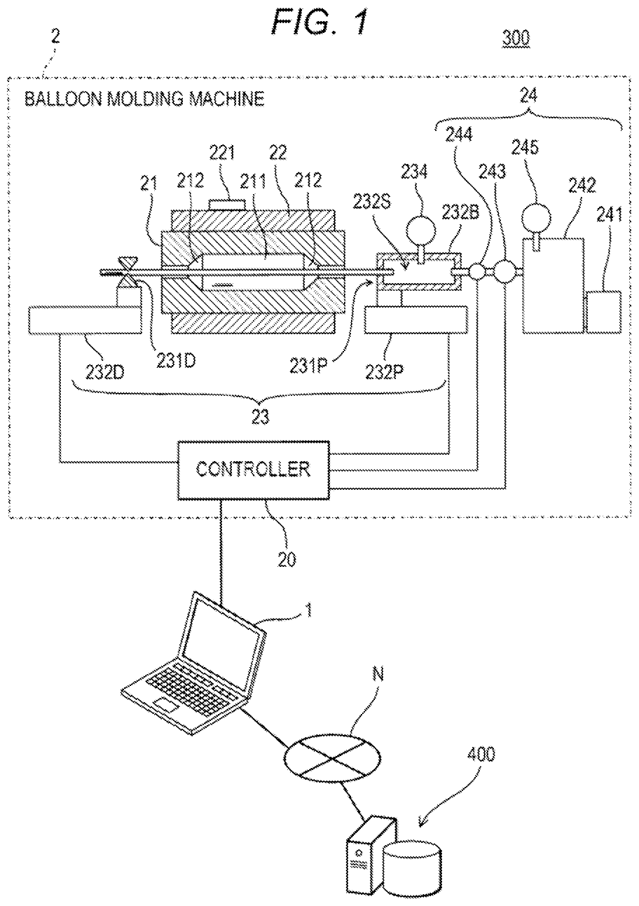

FIG. 1 is a schematic view of a manufacturing system 300 according to a first embodiment. The manufacturing system 300 includes a balloon molding machine 2 and a determination device 1 connectable to a controller 20 of the balloon molding machine 2. In the manufacturing system 300, the determination device 1 can be connected to a production management system 400 in a manufacturing factory of a catheter via a local network N.

As illustrated in FIG. 1, the balloon molding machine 2 includes a tubular molding die 21 for molding a tubular balloon base material called a parison into a balloon, a heater 22 that heats the molding die from the outside, a stretching mechanism 23 that stretches the balloon base material, and a blowing device 24 for inflating the balloon base material. The controller 20 of the balloon molding machine 2 controls these mechanisms.

As illustrated in FIG. 1, the molding die 21 includes a combination of half tubular members 210 each having a cavity face 211 defining the outer diameter shape of the balloon to be formed and tapered faces 212 on the inner side. The shape is not limited to the half tubular shape, and may be divided into a cylinder corresponding to the cavity face 211, and cylinders having the tapered faces 212. Chuck mechanisms 231D and 231P, and cylinders 232D and 232P that constitute the stretching mechanism 23 are disposed outside both ends of the molding die 21. The chuck mechanism 231D on one end side holds one end of the balloon base material so as to crush and seal the one end. The chuck mechanism 231P on the other end side holds the other end of the balloon base material so as not to crush the other end. Both the chuck mechanisms 231D and 231P are secured to the rods of the cylinders 232D and 232P, and move in the length direction of the molding die 21, which is the length direction of the balloon base material, when the cylinders 232D and 232P are operated. The chuck mechanism 231P on the other end side is formed by securing a box member 231B having a space 231S communicating with the blowing device 24 therein to the rod of the cylinder 232P. The blowing device 24 includes: a compressor 241 that feeds a molding fluid (for example, air or nitrogen); a tank 242 that stores the molding fluid to be pressurized; a regulator 243 that reduces the pressure of the high-pressure molding fluid; and a valve 244 that controls the inflow of the molding fluid into the box member 231B. The box member 231B has a pressure gauge 234 for measuring the pressure in the internal space 231S communicating with the inside of the balloon base material.

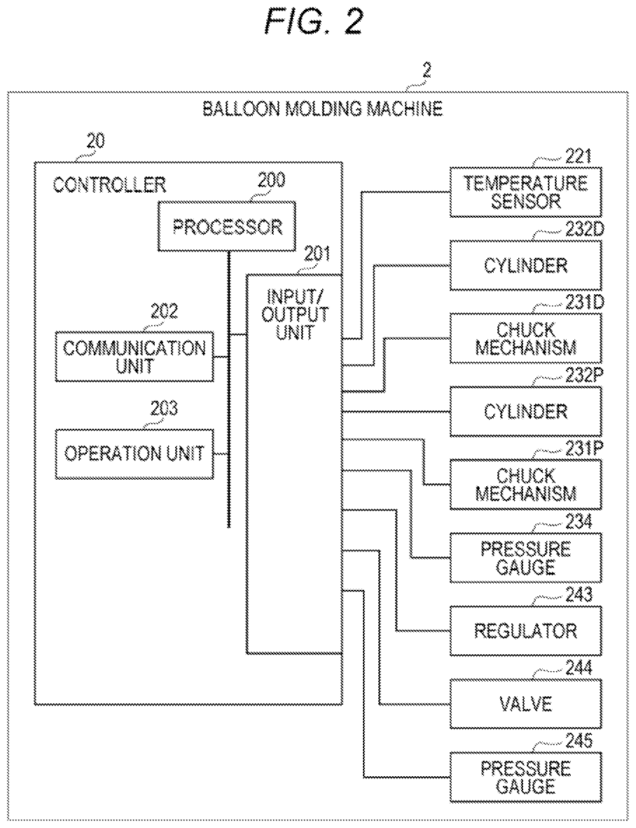

FIG. 2 is a hardware block diagram of the balloon molding machine 2 including the controller 20. The controller 20 is a programmable logic controller, a microcomputer, or the like. The controller 20 includes a processor 200, an input/output unit 201, a communication unit 202, and an operation unit 203.

The processor 200 includes one or more central processing units (CPUs), micro-processing units (MPUs), or the like. The processor 200 may include a graphics processing unit (GPU), a general-purpose computing on graphics processing unit (GPGPU), a tensor processing unit (TPU), or the like. The processor 200 controls the balloon molding machine 2 according to a control program loaded onto a memory such as a random access memory (RAM) (not illustrated) from a read only memory (ROM) or some other memory.

The input/output unit 201 is an interface circuit connected to a temperature sensor 221 that measures the temperature of the heater 22, the cylinders 232D and 232P and the chuck mechanisms 231D and 231P of the stretching mechanism 23, a pressure gauge 245 that measures the pressure in the tank 242 of the blowing device 24, the regulator 243 and the valve 244, the pressure gauge 234 that measures the pressure of the molding fluid in the internal space 231S of the box member 231B, and the like.

The communication unit 202 is a communication interface circuit that performs communication with the determination device 1. The communication unit 202 is a communication interface of a serial bus such as an RS 232C, an RS 422, or an RS 485, for example. The communication unit 202 may be an interface for communication by a local area network (LAN) cable, or may be an interface of a universal serial bus (USB). The communication unit 202 may be a wireless communication device. The communication unit 202 may function like the input/output unit 201 for the processor 200 to exchange data with the determination device 1.

The operation unit 203 includes an output device such as a display, a lamp, and the like, and an input device such as a physical button, a dial, and the like. The operation unit 203 may be a display with a built-in touch panel.

The controller 20 of the balloon molding machine 2 configured as described above controls opening and closing of the components of the molding die 21, heating of the heater 22, movement of the stretching mechanism 23, inflow into the blowing device 24, and pressure adjustment in the blowing device 24, on the basis of an operation performed on the operation unit 203. The controller 20 acquires the temperature of the heater 22 via the input/output unit 201, and controls the display of the operation unit 203 to display the temperature. The controller 20 acquires, via the input/output unit 201, the positions of the rods of the cylinders 232D and 232P, and the opened/closes states of the chuck mechanisms 231D and 231P, and causes the display or the lamp of the operation unit 203 to output the acquired positions and opened/closed states. The controller 20 acquires, via the input/output unit 201, the pressure in the tank 242 of the blowing device 24, the state of the regulator 243, the state of the valve 244, and the pressure in the internal space 231S of the box member 231B, and causes the display or the lamp of the operation unit to output them. Among these pieces of information obtained via the input/output unit 201, the control unit 20 constantly monitors and outputs at least the heater temperature, the positions of the rods, the opened/closed states of the chuck mechanisms 231D and 231P, and the pressure in the box member 231B to the determination device 1 via the communication unit 202.

The controller 20 of the balloon molding machine 2 outputs, from the input/output unit 201, a signal indicating an instruction as to movement of the rods of the cylinders 232D and 232P, switching on/off of the heater 22, opening/closing of the chuck mechanisms 231D and 231P, opening/closing of the valve 244 of the blowing device 24, and adjustment of the regulator 243, in accordance with an operation performed on the operation unit 203 by the operator.

The operator operates the operation unit 203 of the balloon molding machine 2, to heat and pressurize the molding fluid so as to maintain a predetermined temperature and pressure while pouring the molding fluid into the balloon base material inserted into the molding die 21. The operator uses the stretching mechanism 23 to stretch and pressurize both ends of the balloon base material at a predetermined timing, such as when the temperature sufficiently rises, expands the balloon, and stops the stretching mechanism 23 at a predetermined position, to mold the balloon base material. After confirming the expansion of the balloon, the operator cools the molding die 21, and then removes the balloon. The operator trims or cuts both ends of the parison, to produce the balloon.

It has been found that the pressure resistance of the balloon thus produced is affected by the film orientation of the balloon base material and conditions such as temperature, pressure, and stretching rate at the time of molding. It has been further found that the pressure resistance of the resultant balloon can be determined by the parameters obtained at the time of molding the balloon base material. Therefore, in the balloon manufacturing system 300 of the present disclosure, the determination device 1 connected to the balloon molding machine 2 acquires the parameters at the time of molding, and performs determination evaluation on the resistance properties of the balloon. In the description below, a determination process to be performed by the determination device 1 and a control parameter output process to be performed by the determination device 1 are explained.

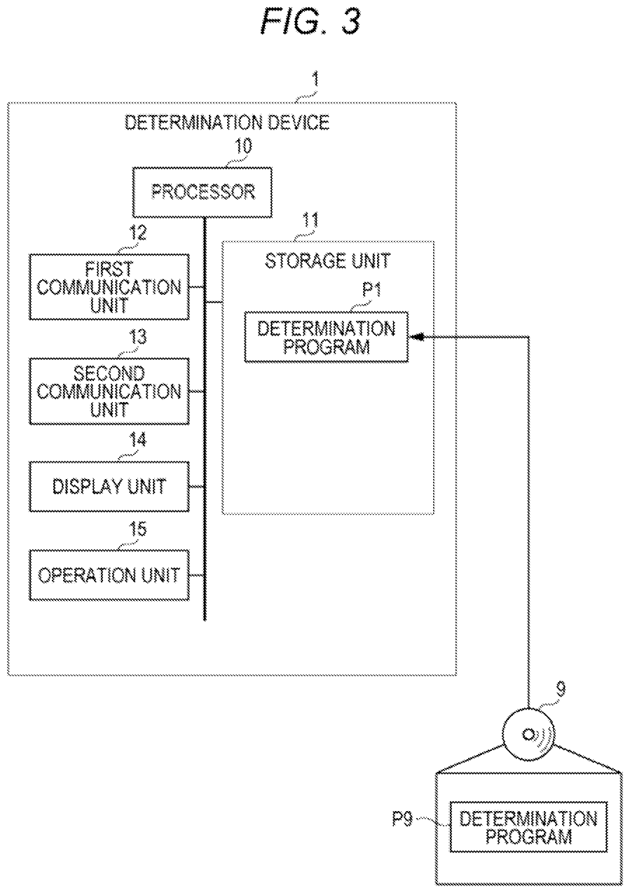

FIG. 3 is a hardware block diagram of the determination device 1. In the description below, the determination device 1 is described as a single device, but a plurality of devices may be connected to each other via a network to perform distributed processing. The determination device 1 may be a portable device such as a smartphone or a tablet terminal.

The determination device 1 includes a processor 10 processor 10, a storage unit 11, a first communication unit 12, a second communication unit 13, a display unit 14, and an operation unit 15. For example, the processor 10 is a CPU and/or a GPU. The processor 10 performs a determination process for the pressure resistance of the balloon manufactured by the balloon molding machine 2, on the basis of a determination program P1 stored in the storage unit 11.

The storage unit 11 is a nonvolatile storage device such as a hard disk drive (HDD), a solid state drive (SSD), or a flash memory.

The storage unit 11 stores data of the distance for comparison to be referred to by the processor 10. The data of the distance for comparison is stored for each type of balloon base material to be machined in the balloon molding machine 2, for each physical property of balloon base materials, for each applied pressure, or for each temperature.

The determination program P1 may be a copy of a determination program P9 stored in a non-transitory storage medium 9 outside the device. The determination program P1 may be distributed by a remote download server, be acquired by the processor 10 via the second communication unit 13, and then be stored into the storage unit 11.

The first communication unit 12 performs communication with the controller 20 of the balloon molding machine 2. The first communication unit 12 is an interface circuit that establishes a connection with the communication unit 202 included in the controller 20. The first communication unit 12 is a communication interface circuit of a serial bus such as an RS 232C, an RS 422, or an RS 485, for example. As described above, the first communication unit 12 may be a LAN interface, a USB interface, or a wireless communication device. The processor 10 controls the first communication unit 12 to receive data from the controller 20 of the balloon molding machine 2. The processor 10 further controls the first communication unit 12, to transmit control data to the balloon molding machine 2.

The second communication unit 13 is a communication interface circuit that performs communication with the production management system 400 in the catheter manufacturing factory via the local network N. The processor 10 controls the second communication unit 13 to acquire, from the production management system 400, data from which physical properties can be determined, such as the type and lot data of the balloon base material to be machined. The processing unit 10 can associate a determination result with data for identifying the individual balloon to be determined, and control the second communication unit 13 to transmit the combined data to the production management system 400. The second communication unit 13 is not necessarily provided, and, for example, an input of the data of the balloon base material may be received via the operation unit 15, a determination result may be stored in the storage unit 11, or only a determination result may be displayed on the display unit 14.

The display unit 14 is a display such as a liquid crystal display (LCD) or an organic electro-luminescence (EL) display. The display unit 14 may be a display with a built-in touch panel, for example. The processor 10 causes the display unit 14 to display text, an image, and the like indicating a result of a determination process to be described later.

The operation unit 15 is a user interface capable of inputting and outputting data under the control of the processor 10. The operation unit 15 is a mouse and a keyboard, for example. The operation unit 15 may be a touch panel containing the display unit 14. The operation unit 15 may be physical buttons. The operation unit 15 may be a sound input unit.

The determination device 1 may further include a sound output unit such as a speaker, and may output a notification sound or a warning sound of a determination result through a process performed by the processor 10. The determination device 1 may be connected to a radio frequency identification (RFID) reader to acquire the ID of the operator or individual identification data of the balloon base material.

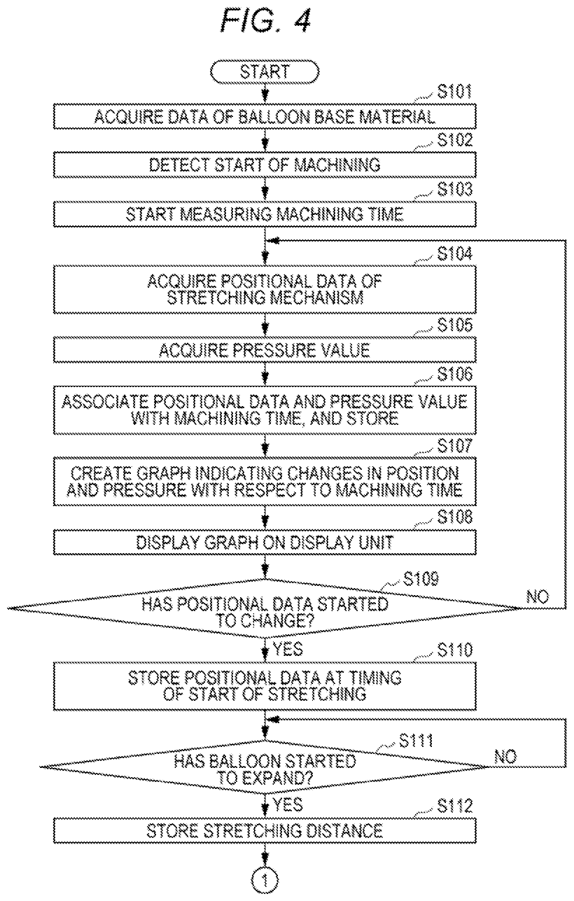

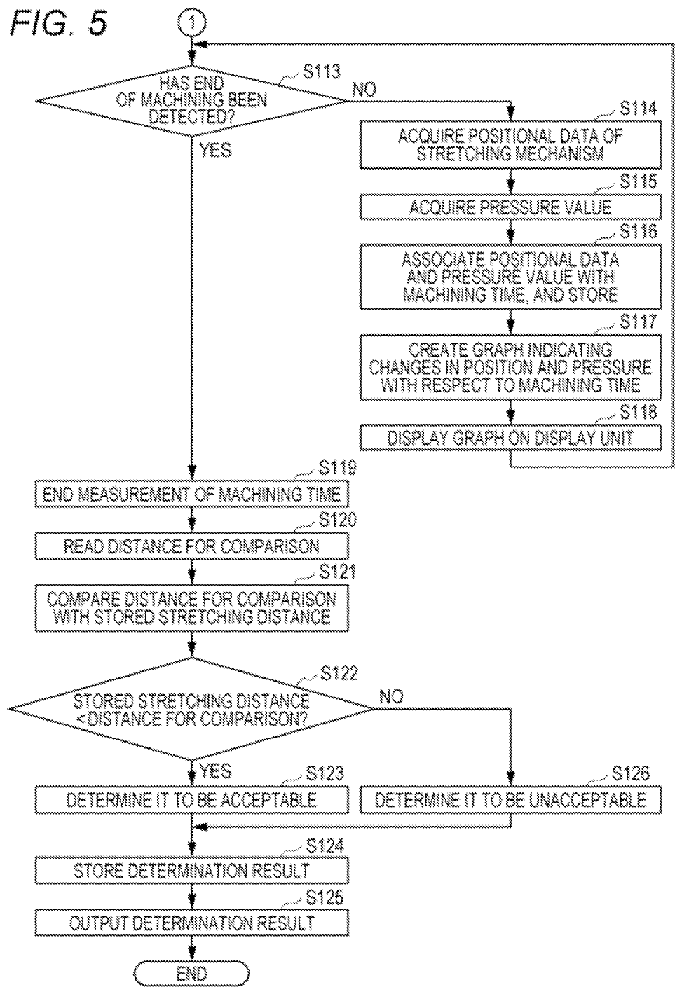

FIGS. 4 and 5 are flowcharts illustrating a determination process to be performed by the determination device 1. The determination device 1 performs the following process each time one balloon base material is machined by the balloon molding machine 2.

The processor 10 acquires data of the balloon base material to be machined (step S101). In step S101, the processor 10 may acquire the type of the balloon base material to be machined by an operation from the operation unit 15, may acquire the type from the production management system 400 via the second communication unit 13, or may read type data from a tag attached to the balloon base material with an RFID reader or the like.

The processor 10 detects the start of machining (step S102). In step S102, the processor 10 may acquire and detect, from the controller 20 of the balloon molding machine 2, a notification of pressing of the start button of the operation unit 203 of the balloon molding machine 2. The processor 10 may acquire and detect, from the controller 20 of the balloon molding machine 2, a change of the heater 22 to an ON-state. The processor 10 may monitor the temperature of the heater 22 from the controller 20 of the balloon molding machine 2, and detect the start of machining from a rise in temperature.

The processor 10 starts measuring the machining time (step S103).

The processor 10 acquires, from the controller 20 of the balloon molding machine 2, positional data of the rods of the cylinders 232D and 232P at both ends of the stretching mechanism 23 (step S104). The processor 10 acquires, from the controller 20 of the balloon molding machine 2, a pressure value measured by the pressure gauge 234 that measures the pressure in the internal space 231S of the box member 231B of the stretching mechanism 23 (step S105). In step S105, the processor 10 may also acquire a pressure value from the pressure gauge 245 of the tank 242 of the blowing device 24.

The processor 10 associates the acquired positional data and pressure value with the machining time, and stores the associated pieces of data in the storage unit 11 (step S106). The processor 10 generates graph data indicating the changes in the positional data and the pressure value with respect to the machining time (step S107), and controls the display unit 14 to display the graph (step S108).

The processor 10 determines whether the positional data has started changing (step S109). In step S109, the processor 10 detects the timing at which the operator starts stretching the balloon by operating the stretching mechanism 23 in the balloon molding machine 2 described above.

If it is determined that the positional data has not started changing (S109: NO), the processor 10 returns the process to step S104, and continues to monitor changes in the positional data and the pressure value with respect to the lapse of the machining time.

If it is determined that the positional data has started changing (S109: YES), the processor 10 stores the positional data at timing of the stretching start (hereinafter referred to as the stretching start position) (step S110), and determines whether the balloon has started expanding (step S111). In step S110, the processor 10 stores positional data indicating the positions of the rods at both ends of the stretching mechanism 23 at the timing of the start of stretching. In step S111, when the balloon starts expanding by stretching and pressurization, the pressure inside the balloon drops, and the pressure in the internal space 231S of the box member 231B of the stretching mechanism 23 communicating with the inside of the balloon also drops. Accordingly, the processor 10 makes determination on the basis of whether the pressure has dropped.

If it is determined that the balloon has not started expanding (S111: NO), the processor 10 returns the process to step S111, and continues the acquisition of the positional data and the pressure value, the measurement of the machining time, the storing of the positional data and the pressure value into the storage unit 11, and the display of the positional data and the pressure value on the display unit 14.

If it is determined that the balloon has started expanding (S111: YES), the processor 10 stores the stretching distance from the stretching start position (step S112). In step S112, the processor 10 adds up the moving distances of the rods at both ends of the stretching mechanism 23 from the stretching position, and stores the result of the addition as the stretching distance.

After that, the processor 10 determines whether the end of the machining has been detected (step S113). If it is determined that the end of the machining has not been detected (S113: NO), the positional data of the rods of the cylinders 232D and 232P at both ends of the stretching mechanism 23 are acquired (step S114), and the pressure value is acquired (step S115). The processor 10 associates the acquired positional data and pressure value with the machining time, and stores the associated pieces of data in the storage unit 11 (step S116). The processor 10 generates graph data indicating the changes in the positional data and the pressure value with respect to the machining time (step S117), and controls the display unit 14 to display the graph (step S118). The display of the graph indicating the changes in the positional data and the pressure value with respect to the machining time on the display unit 14 is continued.

If it is determined that the end of the machining has been detected (S113: YES), the processor 10 ends the measurement of the machining time (step S119). On the basis of the data acquired in step S101, the processor 10 reads, from the storage unit 11, the distance for comparison corresponding to the type of the balloon base material to be machined (step S120). In step S120, the processor 10 may read the distance for comparison corresponding to the physical properties of the balloon base material. In step S120, the processor 10 may read the distance for comparison corresponding to the pressure applied before the pressure drop. This is because the applied pressure may change the stretching distance before the balloon base material starts to expand.

The processor 10 compares the read distance for comparison with the stretching distance stored in step S112 (step S121), and determines whether the stretching distance stored in step S112 is shorter (step S122).

If it is determined that the stored stretching distance is shorter than the read distance for comparison (S122: YES), the processor 10 determines that the pressure resistance of the manufactured balloon is acceptable (step S123). The processor 10 associates identification data of the balloon base material and identification data for identifying the manufactured balloon with the determination result, and stores the identification data (step S124). The processor 10 then controls the display unit 14 to output the determination result (step S125), and ends the determination process.

If it is determined that the stored stretching distance is equal to or longer than the read distance for comparison (S122: NO), the processor 10 determines that the pressure resistance of the manufactured balloon is unacceptable (step S126), moves the process on to step S124, and ends the determination process.

In the processing procedures shown in FIGS. 4 and 5, the processor 10 uses the stretching distance to determine how long it takes to start expansion. The processor 10 may determine whether the time required to start expansion is within a predetermined time.

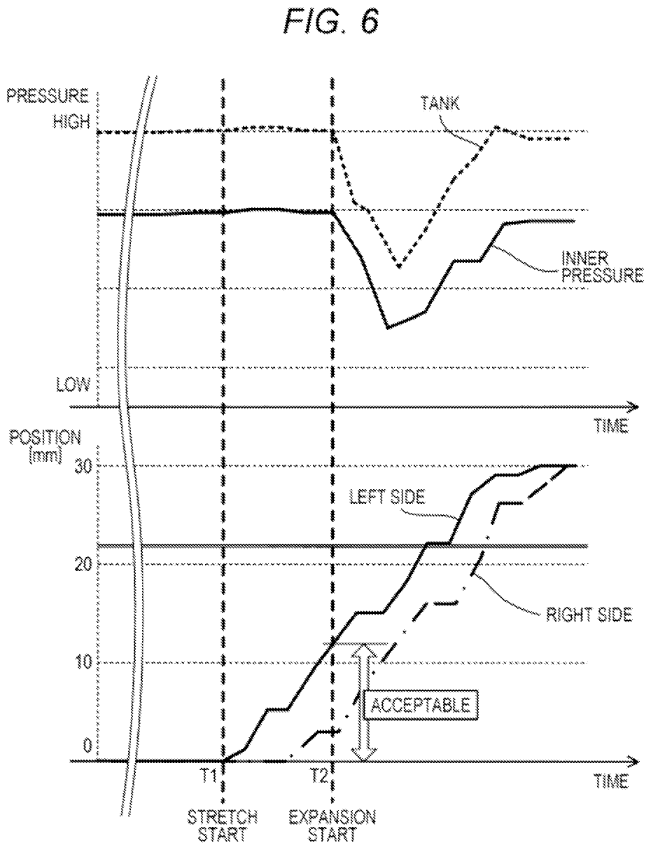

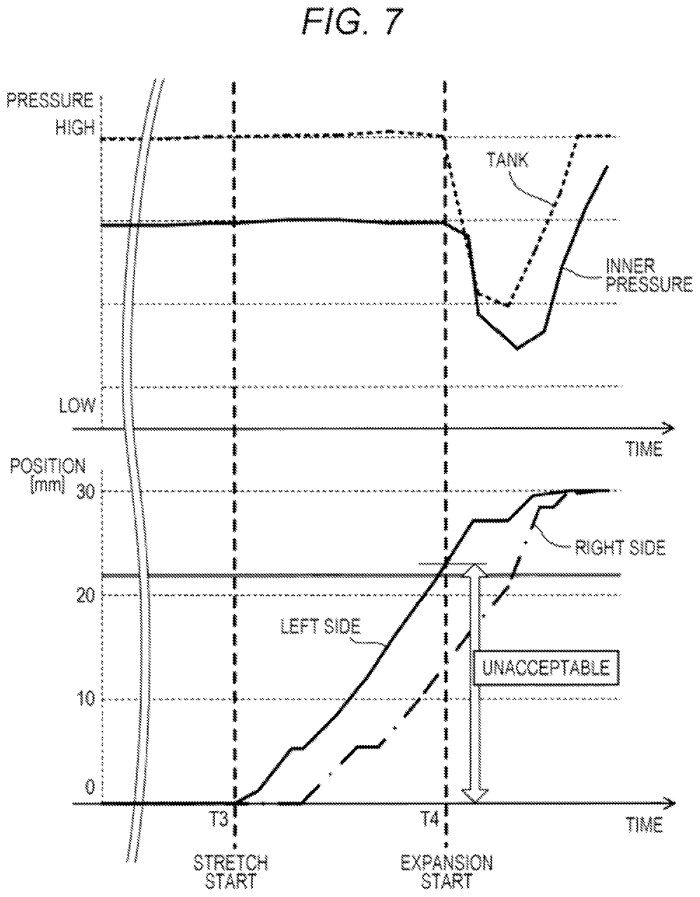

FIGS. 6 and 7 each illustrate an example of the graph displayed on the display unit 14. In both the graph illustrated in FIG. 6 and the graph illustrated in FIG. 7, the abscissa axis indicates the machining time, and the ordinate axis indicates the pressure and the stretching distance. The stretching distance is indicated by the amounts of movement (i.e., the distances of outward movement) of the rods at both ends (i.e., right and left) in the stretching mechanism.

As can be seen from the graph illustrated in FIG. 6, at time T1, the cylinders are actuated to start stretching, and, at time T2, the balloon starts expanding, and the pressure drops. It is recorded that the stretching distance from time T1 to time T2 is 10.1 mm. As can be seen from the graph illustrated in FIG. 7, on the other hand, at time T3, the cylinders are actuated to start stretching, and, at time T4, the balloon starts expanding, and the pressure drops. It is recorded that the stretching distance from time T3 to time T4 is 21.2 mm. In a case where the distance for comparison is 20 mm, a balloon following a course as in the graph illustrated in FIG. 6 during the machining is determined to have a stored stretching distance shorter than the distance for comparison, and is determined to be acceptable. On the other hand, a balloon following a course as in the graph illustrated in FIG. 7 during the machining is determined to have a stored stretching distance equal to or longer than the distance for comparison, and is determined to be unacceptable.

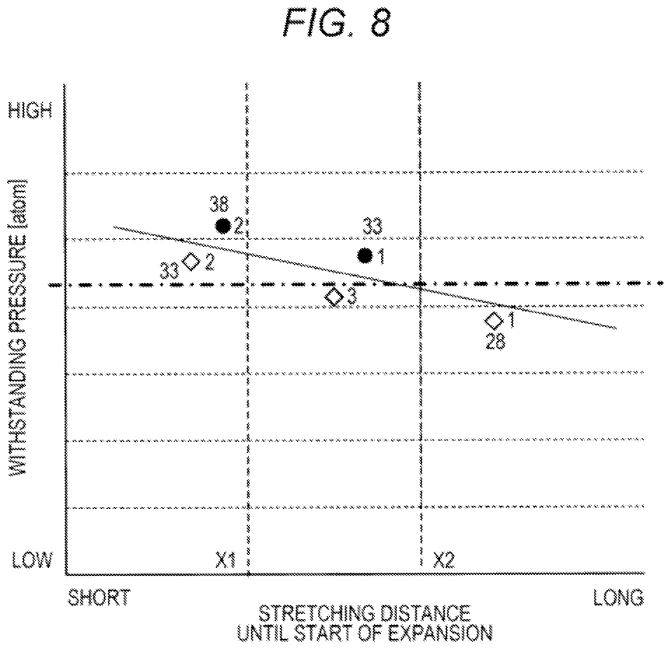

The basis for determining a pressure resistance to be acceptance/unacceptable in the determination in steps S121 and S122 is now described. FIG. 8 is a graph plotting the relationship between the stretching distance before the start of expansion of a balloon and the actual pressure resistance thereof. In FIG. 8, the abscissa axis indicates the stretching distance from the start of stretching to the start of expansion, and the ordinate axis indicates the withstanding pressure based on experiments. In FIG. 8, parameters for balloons of the same lot, which is the same type of balloon base material, are distinguished by the shapes of plotted points. For each plotted point, the numerical value of the pressure of the molding fluid is shown in bar. The plotted position of the black circle with number “1” indicates the pressure resistance of a balloon expanded from a parison of the lot indicated by the black circle with the pressure of the molding fluid set to 33 [bar], and the stretching distance of the balloon at the time of expansion. The plotted position of the black circle with number “2” indicates the pressure resistance of a balloon expanded from a parison of the lot indicated by the black circle with the pressure of the molding fluid set to 38 [bar], and the stretching distance of the balloon at the time of expansion. Likewise, the plotted position of the white rhombus with number “1” indicates the pressure resistance of a balloon expanded from a parison of the lot indicated by the white rhombus with the pressure of the molding fluid set to 28 [bar], and the stretching distance of the balloon at the time of expansion. The plotted position of the white rhombus with number “2” indicates the pressure resistance of a balloon expanded from a parison of the lot indicated by the white rhombus with the pressure of the molding fluid set to 33 [bar], and the stretching distance of the balloon at the time of expansion.

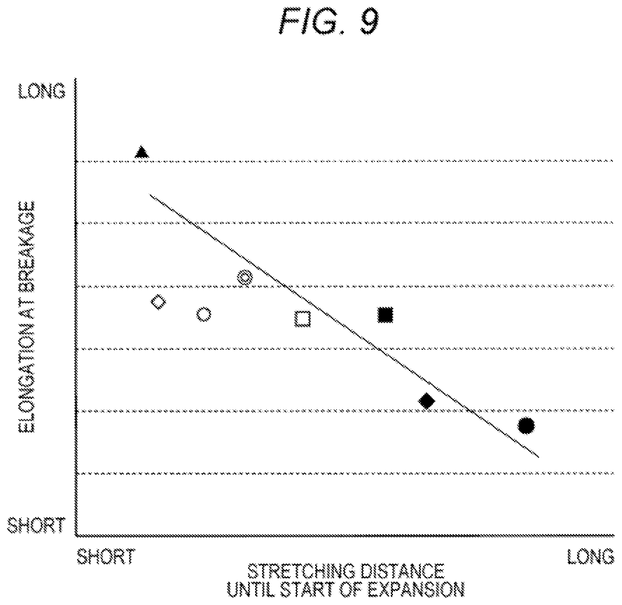

Balloons manufactured by expansion with the pressure of the molding fluid at the same pressure of 33 [bar] from the parison of the lot indicated by the black circle denoted by number “1” and parison of the lot indicated by the white rhombus denoted by numeral “2” have similar pressure resistances. As for the elongation at breakage of a manufactured balloon, however, the balloons manufactured from the parison of a lot (type) indicated by a white rhombus have greater elongation at breakage between the parison of a lot indicated by a black circle and the parison of the lot indicated by the white rhombus (see FIG. 9). Therefore, it is clear that the strength of a balloon cannot be controlled only with pressure. Even if it is guaranteed that the withstanding pressure for the parison of the lots indicated by black circles 1 and 2 is a predetermined value (i.e., the dot-and-dash line in FIG. 8) or higher while the stretching distance before the start of expansion is X2 or shorter, the pressure resistance for the parison of the lots indicated by white rhombuses might be slightly lower than the predetermined value (i.e., the dot-and-dash line in FIG. 8) even in a case where the stretching distance before the start of expansion is shorter than X2 (for example, the white rhombus denoted by number “3”). In this case, for the parison of the lots indicated by white rhombuses, a check may be made to determine whether the stretching distance is shorter than X1.

The reason why a higher pressure resistance is achieved when the stretching distance before the start of expansion is shorter is examined as follows. It is assumed that a pressure resistance is caused by variation in film orientation among a plurality of layers of parison as a balloon base material.

As for the strength observed when the parison is stretched in the length direction, a tensile test in the length direction is conducted on a sample of each lot of parison, to obtain parameters. The elongation at breakage in the parison tensile test cannot be ignored as a parameter for evaluating pressure resistance. However, it has been found that the elongation at breakage in the tensile test and the stretching distance before the start of expansion at the time of machining described above have a correlation to some extent. FIG. 9 is a graph illustrating a correlation between stretching distance before the start of expansion of a balloon and elongation thereof at breakage. The abscissa axis in FIG. 9 indicates the average value of the above stretching distances when balloons were actually manufactured with respect to the parison of each lot. The ordinate axis in FIG. 9 indicates the elongation at breakage obtained in a tensile test conducted on the parison of each lot. The correlation chart in FIG. 9 shows that the elongation at breakage tends to be longer when the stretching distance until expansion is shorter. It is safe to say that the molecular orientation inside the parison is not aligned in the long axis direction of the balloon when the elongation at breakage is large, but the molecular orientation is strong in the long axis direction when the elongation at breakage is small. When the two are compared with each other, it can be seen from FIG. 8 that the pressure for expanding the parison requires to be higher when the elongation at breakage is smaller. The difference in pressure indicates that the rate at which the parison expands varies. That is, in the parison with the larger elongation at breakage, the pressure is low, and therefore, not only molecular orientation in the circumferential direction but also orientation in the long axis direction is applied during expansion. On the other hand, in the parison with the smaller elongation at breakage, expansion due to high pressure weakens the orientation in the long axis direction, resulting in a stronger orientation in the circumferential direction. Since the strength of a plastic film is normally higher in the orientation direction, the orientation strength in the circumferential direction correlates with the pressure resistance of the balloon. Furthermore, even in the case of the parison of the same lot, which is the parison from which the same elongation at breakage can be estimated, the stretching distance during machining might vary with each parison. Since the correlation between the stretching distance before the start of expansion and the pressure resistance during the machining described above is high, the stretching distance before the start of expansion described above can be set as a parameter of high importance in evaluating the pressure resistance.

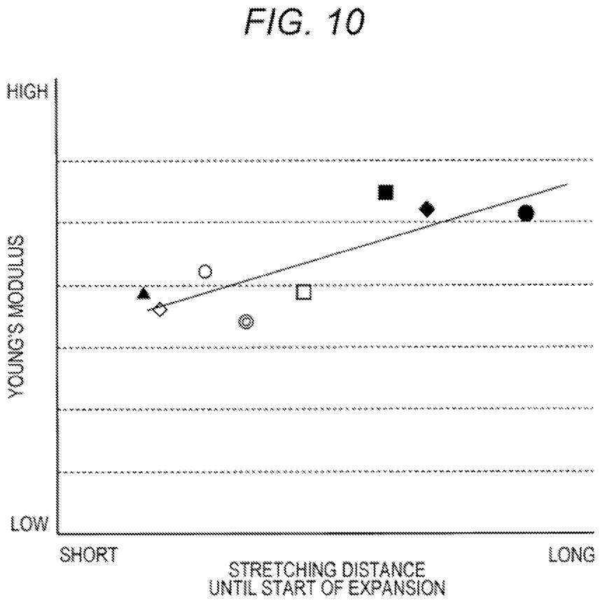

Likewise, for evaluation of variation in film orientation, the Young's modulus of each sample of parisons is measured, and can be and used as a parameter for the parison of each lot. During the machining of the original material tube, measurement with a spectrometer can be performed to calculate an evaluation value regarding the orientation in the length direction, and the evaluation value can be used as a parameter. However, it has been found that the Young's modulus and the stretching distance before the start of expansion at the time of machining described above also have a correlation. FIG. 10 is a graph illustrating a correlation between stretching distance and Young's modulus. The abscissa axis in FIG. 10 indicates the average value of the above stretching distances when balloons were actually manufactured with respect to the parison of each lot, and the ordinate axis indicates the Young's moduli of parison samples of the respective lots. The Young's modulus obtained when a tensile test in the long axis direction is conducted depends on the strength of the molecular orientation in the long axis direction inside the parison. This indicates that expansion in the circumferential direction hardly occurs in a parison having a high Young's modulus as compared with a parison having a low Young's modulus, and a high pressure is required for balloon molding. The difference in pressure indicates that the rate at which the parison expands also varies herein. That is, in the parison with the lower Young's modulus, the pressure is low, and therefore, not only molecular orientation in the circumferential direction but also orientation in the long axis direction is applied during expansion. On the other hand, in a parison having a high Young's modulus, because of expansion by high pressure, molecular orientation due to expansion in the circumferential direction is stronger than that due to stretching in the long axis direction. Since the strength of a plastic film is normally higher in the orientation direction as described above, the orientation strength in the circumferential direction correlates with the pressure resistance of the balloon. Furthermore, even in the case of the balloon base material of the same lot, which is the balloon base material from which the same Young's modulus can be estimated, the stretching distance during machining might vary with each parison. Since the correlation between the stretching distance before the start of expansion and the pressure resistance during the machining described above is high, the stretching distance before the start of expansion described above can be set as a parameter of high importance in evaluating pressure resistance, even with the Young's modulus being taken into consideration.

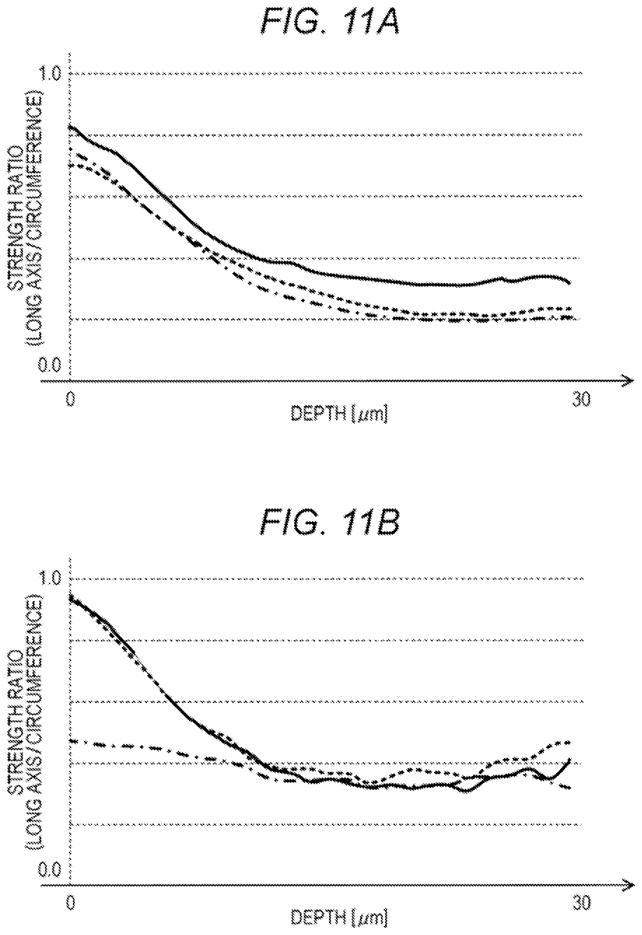

Further, it was experimentally found that balloons having shorter stretching distances before the start of expansion during the machining described above had less variation in balloon strength, regardless of the Young's moduli. Here, the strength is indicated by a ratio between the strength in the balloon length direction and the strength in the circumferential direction. FIGS. 11A and 11B are graphs illustrating strength distribution depending on depth. In both FIGS. 11A and 11B, the abscissa axis indicates the depth from the surface, and the ordinate axis indicates the strength ratio. FIGS. 11A and 11B each illustrate a distribution of the strength ratio measured at each position in the balloon length direction. FIG. 11A illustrates a strength distribution of balloons each having a short stretching distance before the start of expansion, and FIG. 11B illustrates a strength distribution of balloons each having a relatively long stretching distance before the start of expansion. Comparing FIGS. 11A and 11B, it is obvious that, near the surface of the balloon, the strength already varies at the distal end, the central portion, and the proximal end of the balloon. This means that the film orientation varies.

It can be said that it is appropriate to determine whether the pressure resistance of a balloon manufactured by the balloon molding machine is acceptable (i.e., non-defective) or unacceptable (i.e., defective), on the basis of the parameters including the pressure during the machining in the balloon molding machine 2 and the stretching distance by the stretching mechanism. Balloons determined to be unacceptable are not sent to the downstream side in the manufacturing process, so that the quality of the finished product as a balloon catheter can be maintained. Even for an acceptable balloon, the stretching distance before the start of expansion is calculated and stored as an index value representing the pressure resistance of the balloon, to clarify quality guarantee.

### Second Embodiment

In a second embodiment, a determination device 1 determines the pressure resistance of a manufactured balloon, using not only the stretching distance of a stretching mechanism 23 before the start of expansion during machining by a balloon molding machine 2, but also other parameters. The configuration of a balloon catheter manufacturing system 300 of the second embodiment is similar to that of the manufacturing system 300 of the first embodiment, except for details of a determination process to be performed by the determination device 1. Therefore, of the components of the manufacturing system 300 of the second embodiment, the same components as those of the manufacturing system 300 of the first embodiment are denoted by the same reference numerals as those used in the first embodiment, and detailed explanation of them is not made herein.

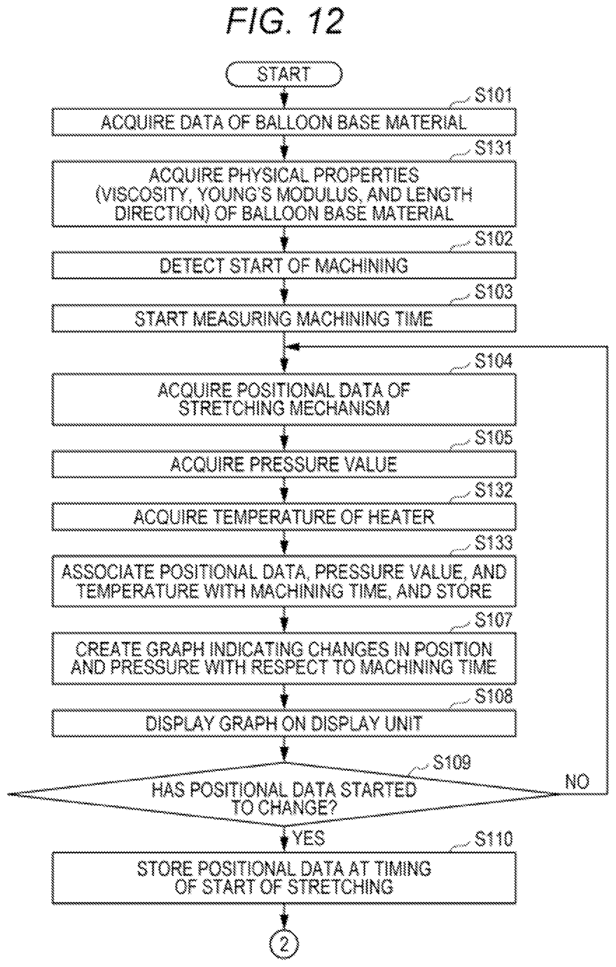

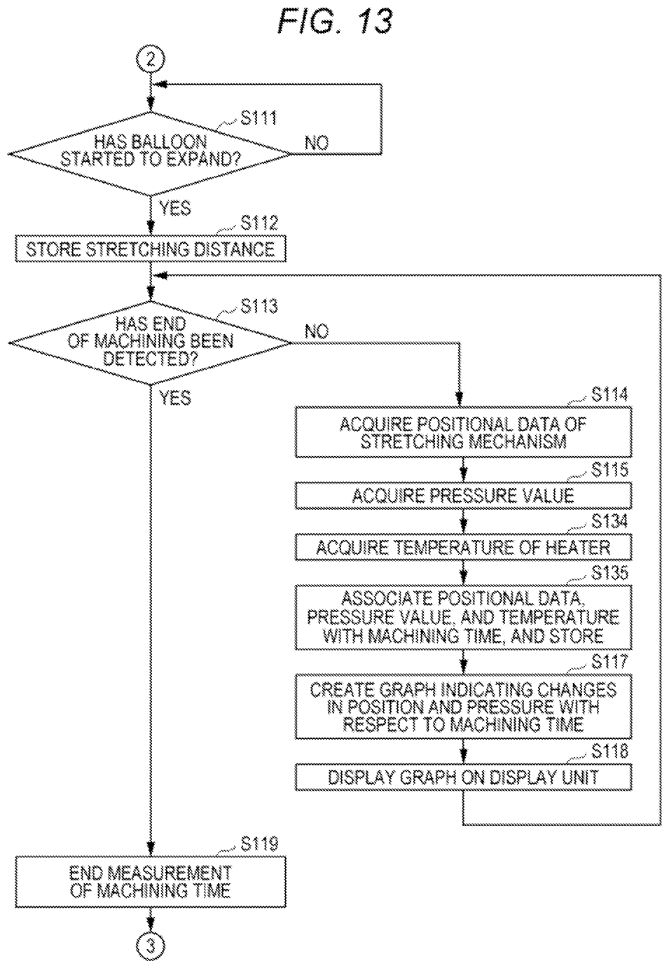

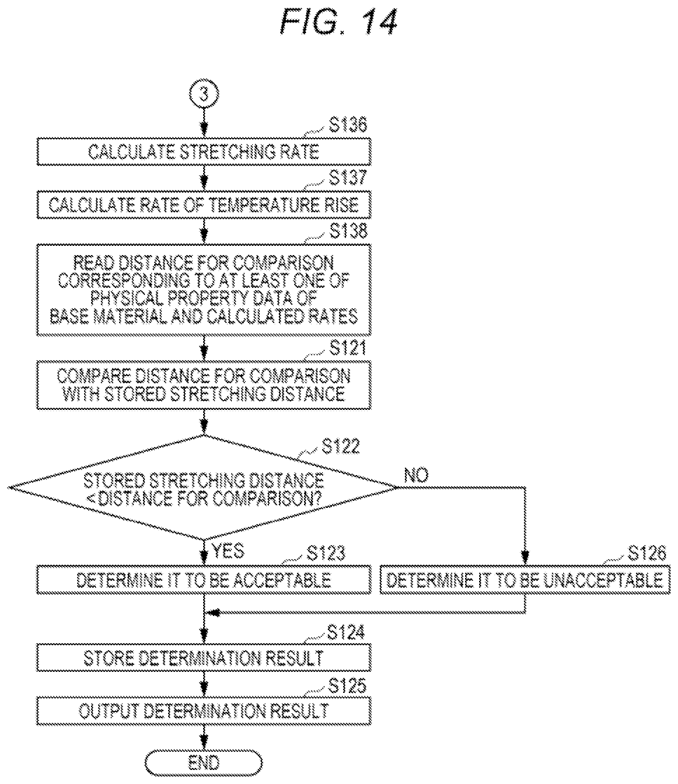

FIGS. 12 to 14 are flowcharts illustrating a determination process to be performed by the determination device 1 according to the second embodiment. Of the processing procedures illustrated in FIGS. 12 to 14, the same procedures as the processing procedures illustrated in FIGS. 4 and 5 of the first embodiment are denoted by the same step numbers as those used therein, and detailed explanation of them is not made herein.

When acquiring the data of the balloon base material to be machined (S101), the processor 10 of the determination device 1 acquires the viscosity, the Young's modulus, and the orientation in the length direction of the material (i.e., resin) of the balloon base material of the same lot or the same type, on the basis of the identification data or the type of the balloon base material (step S131).

As for evaluation of viscosity, Young's modulus, and film orientation in the length direction, measurement is performed on samples of balloon base materials for each lot in advance, and the determination device 1 stores the measurement results in the storage unit 11 so that the measurement results can be referred to.

When the machining in the balloon molding machine 2 is started, the processor 10 acquires the temperature from the temperature sensor 221 of the heater 22, in addition to the positional data (S104) and the pressure value (S105) of the rods of the cylinders 232D and 232P at both ends of the stretching mechanism 23 (step S132). The processor 10 associates the acquired positional data, the acquired pressure value, and the acquired temperature with the machining time, and stores these pieces of data in the storage unit 11 (step S133). The processor 10 generates graph data of the positional data and the pressure value with respect to the machining time (S107), and controls the display unit 14 to display the graph (S108).

In the second embodiment, when stretching is started (S109: YES), the processor 10 also stores the positional data at that timing (S110), and stores the stretching distance at the timing at which expansion of the balloon is started (S112).

The processor 10 acquires the positional data (S114) and the pressure value (S115) of the stretching mechanism 23, acquires the temperature of the heater 22 (step S134), associates the positional data, the pressure value, and the temperature with the machining time, and stores these pieces of data (step S135), until the end of the machining is detected.

In the second embodiment, when the processor 10 detects the end of the machining (S113: YES) and ends the measurement of the machining time (S119), the processor 10 calculates the stretching rate from the start to the end of stretching, on the basis of change in the positional data with respect to the machining time (step S136). The processor 10 calculates the rate of the temperature rise from the start to the end of stretching, on the basis of change in temperature with respect to the machining time (step S137). One of both of the above rates may be calculated.

The processor 10 reads a distance for comparison corresponding to at least one of the following items: data indicating the physical properties of the balloon base material such as viscosity, Young's modulus, and orientation in the length direction acquired until the end of the machining is detected, and the rates calculated in S136 and S137 (step S138). The processor 10 compares the read distance for comparison with the stretching distance stored in step S112 (step S121), and determines whether the stretching distance before the start of expansion is shorter (step S122).

If it is determined that the stretching distance before the start of expansion is shorter (S122: YES), the processor 10 determines that the balloon is acceptable (S123). If it is determined that the stretching distance is equal to or longer than the distance for comparison (S122: NO), the processor 10 determines that the balloon is unacceptable (S126), stores and outputs the determination result (S124 and S125), and ends the process.

In the second embodiment, in step S136, the processor 10 reads the distance for comparison related to the determination as to acceptable/unacceptable, in accordance with the physical properties, temperature, and the like of the balloon base material. The processor 10 of the determination device 1 of the second embodiment performs the determination, using not only the difference between lots (i.e., types), but also the distance for comparison that differs depending on change in temperature, the temperature at the start of stretching, the pressure, and the physical properties.

In step S122, not only the determination based on the stretching distance is performed, but also an index value using physical properties such as a difference in temperature transition and the level of Young's modulus, and an index value based on the stretching distance may be derived, and determination based on the index values may be performed.

### Third Embodiment

As described in the first embodiment and the second embodiment, with the parameters including the pressure during machining by the balloon molding machine 2 and the stretching distance before the start of expansion by the stretching mechanism 23, the pressure resistance of a manufactured balloon can be evaluated as a result. In the second embodiment, evaluation was performed by taking into account the temperature during machining, the stretching rate, and the physical properties of the balloon base material, in addition to the stretching distance before the start of expansion. In a third embodiment, a determination device 1 performs training on the basis of evaluation using a parameter that is the stretching distance before the start of expansion, and a record of the physical property data of the balloon base material. Further, the determination device 1 outputs appropriate control parameters (e.g., temperatures, pressures, stretching rates, etc.) in the balloon molding machine 2 for the balloon base material to be machined.

The configuration of a manufacturing system 300 in the third embodiment is similar to the configuration of the manufacturing system 300 in the first embodiment, except for the processing to be performed by the determination device 1 described later. Therefore, of the manufacturing system 300 of the third embodiment described below, the same components as those of the manufacturing system 300 of the first embodiment are denoted by the same reference numerals as those used in the first embodiment, and detailed explanation of them is not made herein.

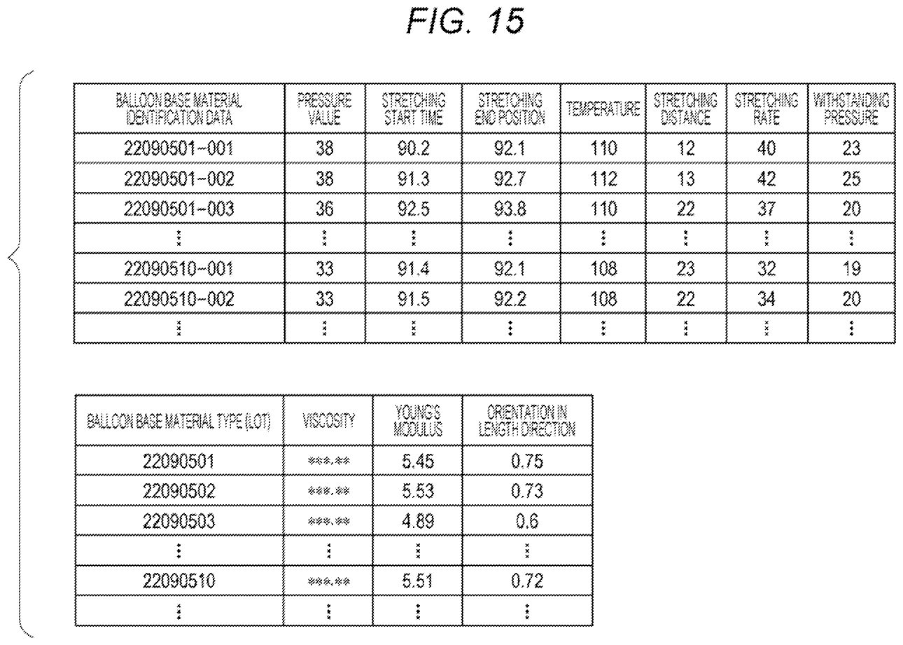

FIG. 15 is a diagram illustrating example contents of data stored in the determination device 1. The determination device 1 can store, from the balloon molding machine 2, the pressure value before the start of expansion and the stretching start position of the balloon base material to be machined, as illustrated in FIG. 15, by the processing procedures illustrated in FIGS. 4 and 5 of the first embodiment. Further, the data illustrated in FIG. 15 may store not only the data of the actual manufacturing of the balloon catheter but also a result (i.e., stretching distance) in a case where the temperature and the pressure in the balloon molding machine 2 are varied.

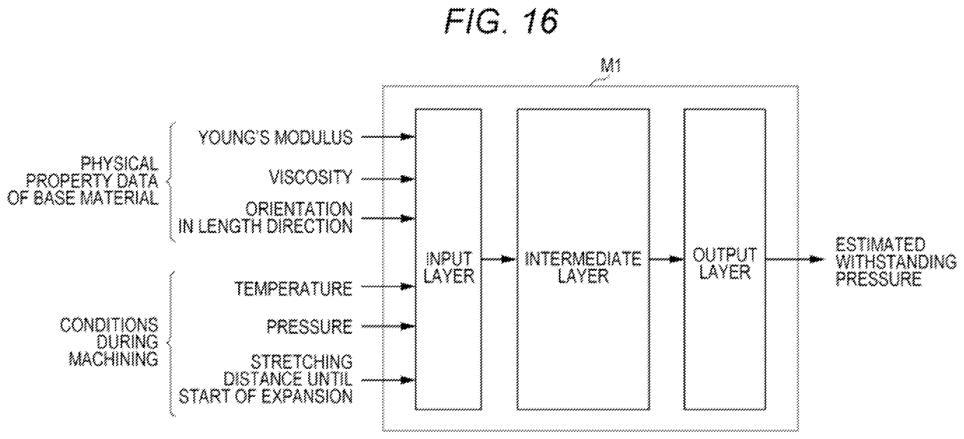

FIG. 15 shows the withstanding pressure measured for each balloon as a manufacturing result. By storing the withstanding pressure indicating the pressure resistance as a result, it is possible to estimate the withstanding pressure from the pressure, the temperature, the stretching distance, and the like during machining in the balloon molding machine 2, using the results illustrated in FIG. 15 as training data. FIG. 16 is a schematic diagram of a trained model M1 that outputs pressure resistance. The trained model M1 is trained so as to output an estimated withstanding pressure in a case where the data of the physical properties of the balloon base material to be machined by the balloon molding machine 2 and the conditions (temperature, pressure, and stretching distance) during the machining are input. The trained model M1 is trained so as to output the withstanding pressure in a case where a numerical value of another item (pressure value, temperature, temperature distance, stretching rate, viscosity, Young's modulus, orientation in length direction, or the like) is input, with the withstanding pressure illustrated in FIG. 15 as the correct data.

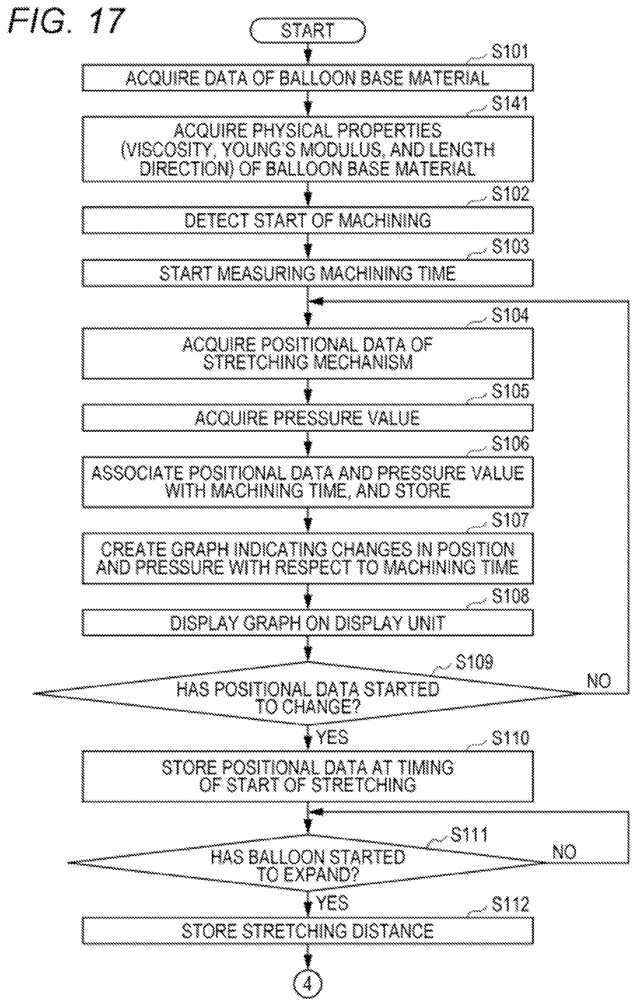

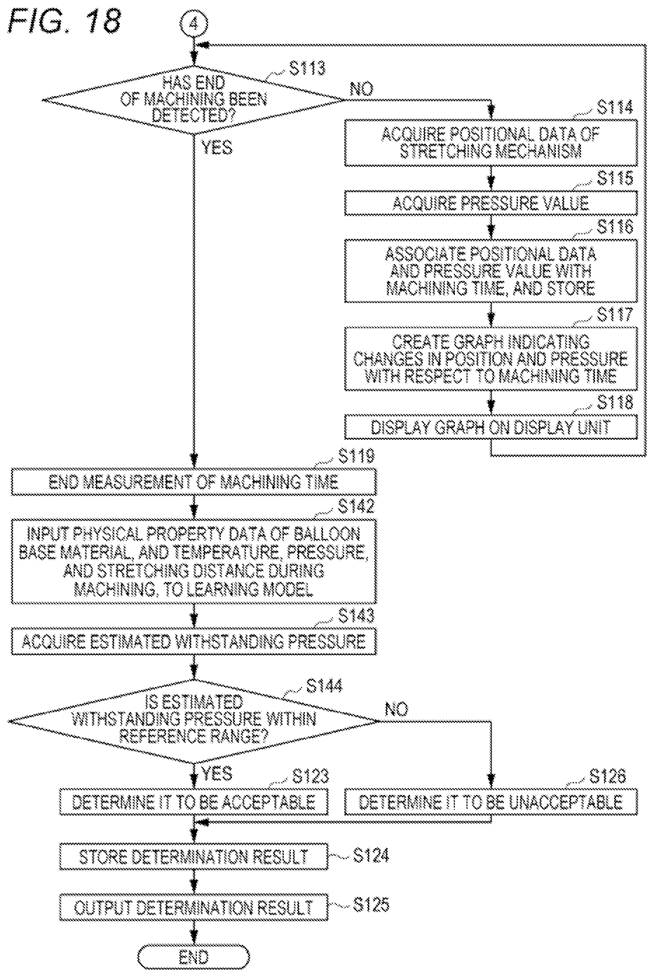

FIGS. 17 and 18 are a flowchart illustrating an example of a determination process using a trained model M1. The determination device 1 performs the following process each time one balloon base material is machined by the balloon molding machine 2. Of the processing procedures illustrated in FIGS. 17 and 18, the same processing procedures as the processing procedures illustrated in FIGS. 4 and 5 of the first embodiment are denoted by the same step numbers as those used therein, and detailed explanation of them is not made herein.

In a case where the trained model M1 is used, when acquiring the data of the balloon base material to be machined (S101), the processor 10 acquires the viscosity, the Young's modulus, and the orientation in the length direction of the material (i.e., resin) of the balloon base material of the same lot or the same type, on the basis of the identification data or the type of the balloon base material (step S141).

When the processor 10 determines that the end of the machining has been detected (S113: YES), and ends the measurement of the machining time (S119), the processor 10 inputs, to the trained model M1, the physical property data of the balloon base material acquired in step S141, and the temperature, pressure, and stretching distance at the time of machining acquired during the machining (step S142).

The processor 10 acquires an estimated withstanding pressure output from the trained model M1 (step S143), and determines whether the estimated withstanding pressure is within a reference range of a balloon (step S144). If it is determined that the estimated withstanding pressure is within the reference range (S144: YES), the processor 10 determines the withstanding pressure to be acceptable (S123). If it is determined that the estimated withstanding pressure is outside the reference range (S144: NO), the processor 10 determines the withstanding pressure to be unacceptable (S126).

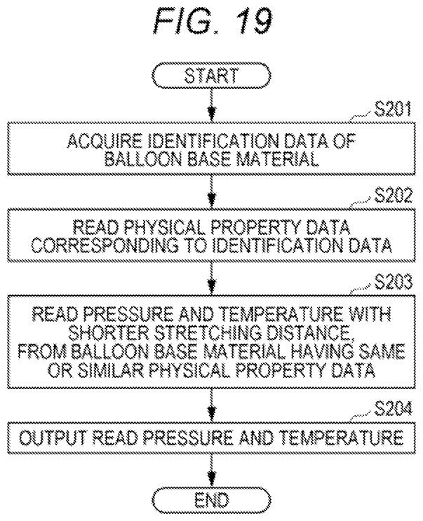

In the third embodiment, a table formed by statistically gathering the data shown in FIG. 15 is used, and control parameters (i.e., temperature, how to change temperature, and pressure) for the balloon molding machine 2 are further determined. FIG. 19 is a flowchart illustrating a method for determining the control parameters by the determination device 1. The determination device 1 performs the following process each time the balloon molding machine 2 starts machining one balloon base material.

The processor 10 acquires identification data (e.g., a lot number) of the balloon base material to be machined (step S201). The processor 10 reads the physical property data corresponding to the identification data from the table stored in the storage unit 11 (step S202). For the balloon base materials having the same or similar physical property data, the processor 10 reads, from the table, the pressure and temperature of a record having a shorter stretching distance before the start of expansion (step S203).

The processor 10 outputs the read pressure and temperature to the display unit 14 and the balloon molding machine 2 (step S204), and ends the process. Thus, in the balloon molding machine 2, the controller 20 uses the output pressure and temperature to control the heater 22 and the stretching mechanism 23 to manufacture a balloon. When balloon manufacturing in the balloon molding machine 2 is performed, the processor 10 also performs the processing procedures illustrated in FIGS. 4 and 5 of the first embodiment.

Accordingly, the temperature and the pressure with which the stretching distance before the start of expansion might be shorter can be utilized for the next machining, on the basis of the past results of machining performed by the balloon molding machine 2. The determination device 1 may output how to change the temperature including the temperature with respect to the machining time. The determination device 1 may output the stretching rate.

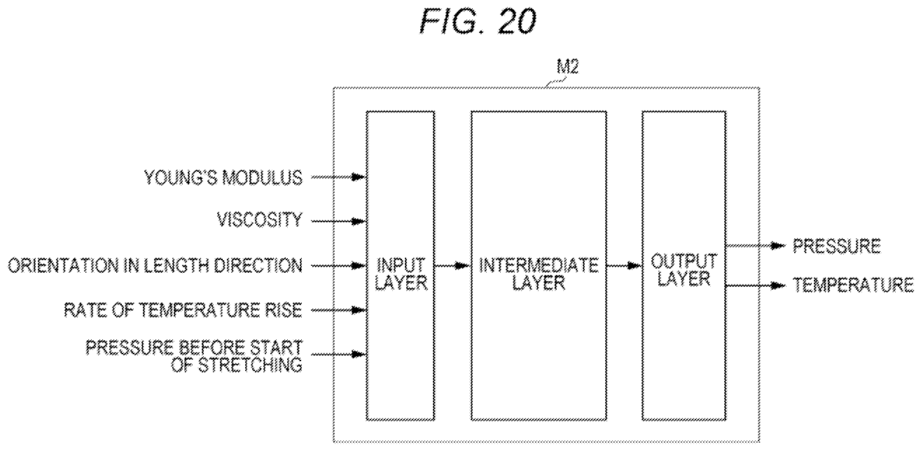

The determination device 1 of the third embodiment may use the data illustrated in FIG. 15 as training data to create and use a trained model M2 trained to output control parameters (i.e., temperature, how to change temperature, and pressure), in a case where the physical property data of the balloon base material to be machined is input by the balloon molding machine 2. FIG. 20 is a schematic diagram of the trained model M2 that outputs control parameters. The trained model M2 includes an input layer that receives physical property data, an intermediate layer that performs calculation, and an output layer that outputs the pressure and temperature in the balloon molding machine 2 as control data. The physical property data includes values indicating the Young's modulus, the resin viscosity, and the orientation in the length direction shown in FIG. 15, for example. In addition to that, the input data may include the temperature before the start of stretching or the rate of temperature rise, and the pressure that has been set before the start of stretching, as illustrated in FIG. 20.

The reinforcement training may be performed on the trained model M2 so that a higher evaluation is obtained when the stretching distance before the start of expansion is shorter.

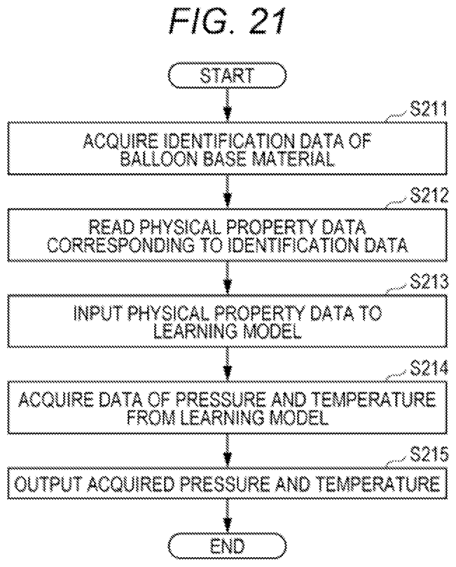

FIG. 21 is a flowchart illustrating a method for determining control parameters using the trained model M2 according to the third embodiment. The determination device 1 performs the following process each time the balloon molding machine 2 starts machining one balloon base material.

The processor 10 acquires identification data (e.g., a lot number) of the balloon base material to be machined (step S211). The processor 10 reads the physical property data corresponding to the identification data, from the table stored in the storage unit 11 (step S212).

The processor 10 inputs at least the read viscosity, Young's modulus, and orientation in the length direction to the trained model M2 (step S213), and acquires pressure and temperature data that is output from the trained model M2 (step S214). The processor 10 outputs the acquired pressure and temperature to the display unit 14 and the balloon molding machine 2 (step S215), and ends the process. Thus, in the balloon molding machine 2, the controller 20 uses the output pressure and temperature to control the heater 22 and the stretching mechanism 23 to manufacture a balloon. When balloon manufacturing in the balloon molding machine 2 is performed, the processor 10 also performs the processing procedures illustrated in FIGS. 4 and 5 of the first embodiment.

As described above, if manufacturing conditions for shortening the stretching distance are derived on the basis of the strength of the correlation between the data of the stretching distance from the start of stretching to the start of expansion after heating and pressurization in the balloon molding machine 2 and the pressure resistance, it is expected that balloons maintaining a high pressure resistance can be manufactured.

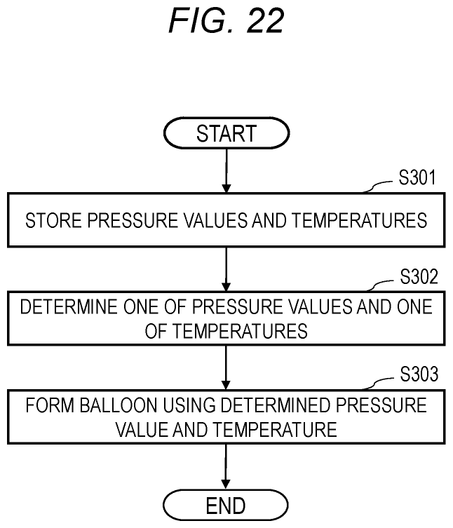

FIG. 22 is a flowchart illustrating a method for manufacturing a balloon for a medical catheter. The determination device 1 and the balloon molding machine 2 perform the method to manufacture a balloon from a target balloon base material.

First, the processor 10 of the determination device 1 acquires and stores in the storage unit 11 a plurality of pairs of pressure value and temperature, which have been used by the balloon molding machine 2 to form balloons from various balloon base materials (step S301). The processor 10 may acquire such data via a network. The processor 10 then determines and selects one of the pressure values and one of the temperatures at which an elongation amount of one of the balloon base materials, which has physical properties that are identical or substantially identical to those of the target balloon base material, is less than a particular value (step S302). The processor 10 may determine a pressure value and temperature at which an elongation time from a start of the elongation of the balloon base material to a start of expansion thereof, is less than a particular value. Subsequently, the processor 10 controls the balloon molding machine 2 to form a balloon using the determined pressure value and temperature from the target balloon base material (step S303).

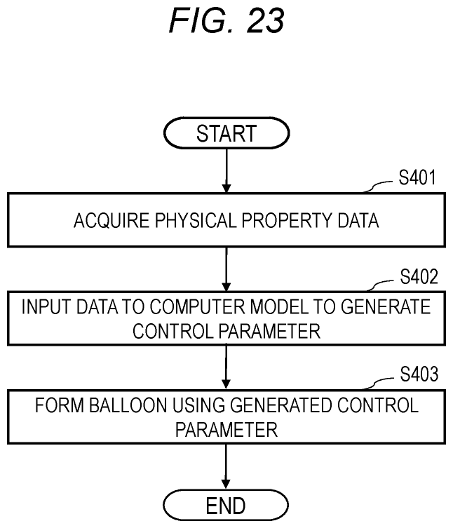

FIG. 23 is a flowchart illustrating another method for manufacturing a balloon for a medical catheter. The determination device 1 and the balloon molding machine 2 perform the method to manufacture a balloon from a target balloon base material.

First, the processor 10 of the determination device 1 acquires physical property data indicating physical properties of the target balloon base material (step S401). The physical property data may be input to the determination device 1 through the operation unit 15, or may be acquired through a network. Next, the processor 10 inputs the physical property data to a computer model, which has been trained to generate a control parameter for controlling the balloon molding machine 2 in response to an input of physical property data of a particular balloon base material (step S402). The model can be trained as described above with reference to FIG. 16. Subsequently, the processor 10 controls the balloon molding machine 2 to form a balloon from the target balloon base material using the control parameter generated by the computer model (step S403).

The embodiments described above are illustrative in all respects and are not restrictive. The scope of the present invention is defined by the claims, and includes meanings equivalent to the claims and all modifications within the scope.

What is claimed is:

1. A method for determining that a balloon for a medical catheter is defective, the method comprising:
in a machining process of elongating a balloon base material with an elongation mechanism while applying pressure therein and heat thereto, monitoring a pressure value of the applied pressure and monitoring a movement amount of the elongation mechanism or a time elapsed after a start of the machining process;
detecting a start of expansion of the balloon base material based on the pressure value;
calculating:
an elongation amount of the balloon base material immediately before the start of expansion of the balloon base material based on the movement amount of the elongation mechanism, or
an elongation time from the start of the machining process to the start of expansion of the balloon base material based on the elapsed time;
determining a pressure resistance of a balloon formed from the balloon base material based on the calculated elongation amount or elongation time; and
determining whether the balloon is defective based on the determined pressure resistance thereof.

2. The method according to claim 1, wherein whether the balloon is defective is determined further based on physical property data including at least one of a viscosity, a Young's modulus, and a film orientation of the balloon base material.

3. The method according to claim 1, further comprising:
storing a reference elongation amount corresponding to the balloon base material before the start of the machining process, wherein
determining whether the balloon is defective includes comparing the calculated elongation amount with the reference elongation amount.

4. The method according to claim 3, wherein the balloon is determined to be defective when the calculated elongation amount is greater than or equal to the reference elongation amount.

5. The method according to claim 1, further comprising:
storing a reference elongation time corresponding to the balloon base material before the start of the machining process, wherein
determining whether the balloon is defective includes comparing the calculated elongation time with the reference elongation time.

6. The method according to claim 5, wherein the balloon is determined to be defective when the calculated elongation time is greater than or equal to the reference elongation time.

7. The method according to claim 1, wherein whether the balloon is defective is determined further based on a temperature of a molding die in which the balloon is formed from the balloon base material.

8. The method according to claim 1, wherein determining the pressure resistance includes inputting physical property data of the balloon base material and the calculated elongation amount or elongation time to a computer model that has been trained to generate an estimated pressure resistance value of the balloon formed from the balloon base material.

9. The method according to claim 1, further comprising:
displaying a determination result indicating whether the balloon is defective.

10. The method according to claim 9, further comprising:
during the machining process, generating graph data indicating a graph of a relationship between the elapsed time and each of the pressure value and the movement amount of the elongation mechanism, and displaying the graph.

11. A determination device for determining that a balloon for a medical catheter is defective, the determination device comprising:
an interface circuit connectable to a balloon molding machine configured to perform a machining process of elongating a tubular balloon base material while applying pressure therein using an elongation mechanism under heat; and
a processor configured to:
when the balloon molding machine is performing the machining process, acquire a pressure value of the applied pressure and acquire a movement amount of the elongation mechanism or a time elapsed after a start of the machining process,
detect a start of expansion of the balloon base material based on the pressure value,
calculate:
an elongation amount of the balloon base material immediately before the start of expansion of the balloon base material based on the movement amount of the elongation mechanism, or
an elongation time from the start of the machining process to the start of expansion of the balloon base material based on the elapsed time,
determine a pressure resistance of a balloon formed from the balloon base material based on the calculated elongation amount or elongation time, and
determine whether the balloon is defective based on the determined pressure resistance thereof.

12. The determination device according to claim 11, wherein whether the balloon is defective is determined further based on physical property data including at least one of a viscosity, a Young's modulus, and a film orientation of the balloon base material.

13. The determination device according to claim 11, wherein the processor is configured to:
store in a memory a reference elongation amount corresponding to the balloon base material before the start of the machining process, and
determine whether the balloon is defective by comparing the calculated elongation amount with the reference elongation amount.

14. The determination device according to claim 13, wherein the balloon is determined to be defective when the calculated elongation amount is greater than or equal to the reference elongation amount.

15. The determination device according to claim 11, wherein the processor is configured to:
store in a memory a reference elongation time corresponding to the balloon base material before the start of the machining process, and
determine whether the balloon is defective by comparing the calculated elongation time with the reference elongation time.

16. The determination device according to claim 15, wherein the balloon is determined to be defective when the calculated elongation time is greater than or equal to the reference elongation time.

17. The determination device according to claim 11, wherein whether the balloon is defective is determined further based on a temperature of a molding die for forming the balloon from the balloon base material.

18. A non-transitory computer readable medium storing a program causing a computer to execute a method for determining a defective balloon manufactured for a medical catheter, the method comprising:
in a machining process of elongating a tubular balloon base material while applying pressure therein by an elongation mechanism under heat, monitoring a pressure value of the applied pressure and monitoring a movement amount of the elongation mechanism or a time elapsed after a start of the machining process;
detecting a start of expansion of the balloon base material based on the pressure value;
calculating:
an elongation amount of the balloon base material immediately before the start of expansion of the balloon base material based on the movement amount of the elongation mechanism, or
an elongation time from the start of the machining process to the start of expansion of the balloon base material based on the elapsed time;
determining a pressure resistance of a balloon formed from the balloon base material based on the calculated elongation amount or elongation time; and
determining whether the balloon is defective based on the determined pressure resistance thereof.

19. A method for manufacturing a balloon for a medical catheter, the method comprising:
storing a plurality of pairs of pressure value and temperature, wherein each of the pairs has been used in forming a balloon in a balloon molding machine by elongating a first balloon base material while applying pressure therein and heat thereto;
determining one of the pressure values and one of the temperatures at which an elongation amount of the first balloon base material or an elongation time from a start of elongation of the first balloon base material to a start of expansion thereof is less than a particular value; and
forming a balloon in the balloon molding device using the determined pressure value and temperature from a second balloon base material having physical properties that are identical or substantially identical to those of the first balloon base material.

20. A method for manufacturing a balloon for a medical catheter, the method comprising:
acquiring physical property data indicating physical properties of a balloon base material to be used for manufacturing a balloon;
inputting the acquired physical property data to a computer model that has been trained to generate a control parameter for controlling a balloon molding machine from physical property data of a balloon base material; and
forming a balloon from the balloon base material with the balloon molding machine using the control parameter that is generated by the computer model.
